(19) United States
(12) Patent Application Publication
(10) Pub. No.: US 2025/0121146 A1
(43) Pub. Date: Apr. 17, 2025

(54) DISPLAY OF A PRE-SET STARTING DOSE OF NO ON A MEDICAL GAS SUPPLY APPARATUS

(71) Applicant: INOSYSTEMS, Antony (FR)

(72) Inventors: Yann BLANDIN, Paris (FR); Frederic MARCHAL, Wissous (FR); Mary SCHMITT, Bagneux (FR); Thierry BOULANGER, Philadelphia, PA (US)

(21) Appl. No.: 18/912,945

(22) Filed: Oct. 11, 2024

(30) Foreign Application Priority Data
Oct. 12, 2023 (FR) 2310973

(51) Int. Cl. A61M 16/00; A61M 16/10; A61M 16/20

(52) U.S. Cl. CPC A61M 16/024; A61M 16/10; A61M 16/202

(57) ABSTRACT

The invention relates to an apparatus (1) for supplying an NO-containing gas, comprising at least one internal passage (6) with valve means (7) for conveying a flow of NO-containing gas; a graphic display (4), and control means (8) with a microprocessor (9) cooperating at least with the valve means (7) in order to control the supply of gas. Storage means (70) are provided for storing a pre-set non-zero dose of NO (NO_{mém}). The control means (8) control a default display (at 71), on the graphic display (4), of the pre-set starting dose of NO (NO_{mém}) that has been stored.

### CROSS REFERENCE TO RELATED APPLICATIONS

This application claims the benefit of priority under 35 U.S.C. § 119 (a) and (b) to French Patent Application No. 2310973, filed Oct. 12, 2023, the entire contents of which are incorporated herein by reference.

### BACKGROUND

The invention relates to an apparatus or device for supplying and monitoring a medical gas containing nitric oxide (NO), in particular an NO/N₂ gas mixture, commonly referred to as an NO supply apparatus, used to treat one or more persons suffering from acute pulmonary arterial hypertension, and configured to display, upon start-up of the apparatus, a stored dose of NO, called a “default” dose, chosen by the medical personnel and pre-recorded, also called a “starting” dose.

Inhaled nitric oxide (NOi) is a standard of care for treating persons, i.e. patients, suffering from acute pulmonary arterial hypertension. When inhaled, NO dilates the pulmonary vessels and increases oxygenation by improving gas exchange. These properties are used to treat various medical conditions such as persistent pulmonary hypertension of the newborn (PPHN), acute respiratory distress syndrome (ARDS) observed mainly in adults, or pulmonary hypertension (PH) in heart surgery, observed in adults or children, as described in particular by EP-A-560928, EP-A-1516639 and U.S. Pat. No. 10,201,564.

Implementation of NOi treatment usually involves one or more NO/N₂ cylinders (or an NO generator), an apparatus for supplying and monitoring NO, a medical ventilator, and a patient kit comprising a patient circuit and respiratory interface, such as a tracheal intubation tube, or other elements, such as a gas humidifier or the like.

Usually, a small quantity of gaseous NO (i.e. a few ppm vol.), diluted in nitrogen (N₂), is injected and diluted in a gas flow containing oxygen, typically at least 20% vol. of oxygen (O₂), such as an N₂/O₂ mixture or air, or even pure oxygen, which is conveyed through the patient circuit of a gas supply installation, and the final gas mixture obtained, containing NO and oxygen, is then inhaled by the patient.

The final concentration or dose of NO, which corresponds to a dosage, is determined by the doctor or the like, or it is set by a medical protocol validated by the medical team. In general, it is between 1 and 80 ppm by volume (ppmv), typically of the order of 10 to 20 ppmv, depending on the population treated, i.e. neonates, children, adolescents or adults, and on the disease to be treated, in the final NO/N₂/O₂ gas administered by inhalation to the patient in question, that is to say after injection of the NO/N₂ mixture into the oxygen-containing gas flow (i.e. approximately >20% vol.) conveyed by the patient circuit.

The desired concentration or dose of NO is usually set, adjusted or selected by the healthcare provider, such as a doctor, a nurse or the like, at the start of a patient's treatment procedure and, in an emergency, at the onset of treatment, that is to say after switching on the NO supply apparatus.

To do this, the NO supply apparatus displays a zero “starting” dose or concentration, that is to say an NO concentration of 0 ppm, and the healthcare provider increments the dose in increments or directly enters/selects a desired “starting” dose, for example 10, 15 or 20 ppm, which will then be used by the NO supply apparatus to provide a suitable NO flow rate, i.e. an NO/N₂ gas mixture flow rate, for achieving this set “starting” dose in the final gas mixture administered by inhalation to the patient to be treated.

However, in practice, errors in NO dosage sometimes occur due to errors in setting or fixing the dose, because the medical personnel need to act urgently when starting an NO treatment and may therefore make a mistake when entering or selecting the dose of NO to be administered at the start.

It will be understood that a dosing error may cause a risk to the patient who does not receive the desired dosage, that is to say an appropriate concentration or dose, in particular a risk of an overdose if the level entered is too high or, by contrast, a risk of an under-dose which can then lead to absence of a therapeutic effect due to the dose being insufficient and ineffective.

A problem is therefore to be able to increase the safety of NO treatment by avoiding the NO dose setting errors that can occur at the beginning of treatment, that is to say the errors associated with the “starting” dose of NO.

### SUMMARY

One solution of the invention relates to an apparatus or device for supplying an NO-containing gas that can be used to treat a patient, such as an NO/N₂ gas mixture, comprising:
at least one internal passage for conveying a flow of NO-containing gas,
valve means arranged on said at least one internal passage,
a graphic display, and
microprocessor-based control means cooperating at least with the valve means in order to control the supply of gas, in particular the flow rate of gas in at least a part of said internal passage.

Moreover, the apparatus or device of the invention comprises:
storage means for storing a pre-set non-zero dose (i.e. a concentration) of NO (NO_{mém}) corresponding to what is called an NO starting concentration to be administered by inhalation to a patient at the initiation of treatment by inhaled NO,
the control means are configured to control a display, on the graphic display, of the pre-set dose of NO (NO_{mém}) that has been stored,
and the graphic display is configured to present, before any initiation of the treatment by inhaled NO, a default display of said pre-set dose of NO (NO_{mém}) that has been stored.

In other words, according to the invention, when the apparatus is started up or switched on, that is to say as soon as it is switched on by actuation, for example, of a start (On/Off) button or key, the apparatus is configured to display a stored default non-zero NO dose corresponding to the non-zero NO concentration that will be administered to the patient at the start of treatment, typically after the user, i.e. the healthcare provider, such as doctor or the like, presses a start-of-treatment key, typically a virtual key displayed on the display screen of the apparatus. This avoids the need for the healthcare provider to enter this dose and therefore reduces the risk of errors and the abovementioned drawbacks.

Depending on the embodiment considered, the apparatus of the invention can comprise one or more of the following features:
the stored pre-set dose of NO (NO_{mém}) is the only NO dose displayed when the apparatus is started (i.e. when the apparatus is switched on), that is to say it is displayed on the display screen automatically and without any possible alternative, i.e. before any initiation of treatment with NO.
the stored pre-set dose of NO (NO_{mém}) is displayed before any administration of NO to the patient commences.
the stored pre-set dose of NO (NO_{mém}) is displayed at the moment the apparatus is started up or switched on, but before any start of administration of NO by the apparatus.
the stored pre-set dose of NO (NO_{mém}) is displayed immediately when the apparatus is started up or switched on.
the stored pre-set dose of NO (NO_{mém}) is not an alarm threshold.
the stored pre-set dose of NO (NO_{mém}) corresponds to the desired NO concentration in the gas mixture to be administered to the patient, i.e. the dosage to be observed in the context of treatment with supply of inhaled NO to said patient. Typically, the gas mixture administered, also called the final mixture, contains NO, nitrogen (N₂) and oxygen (O₂), or even other compounds, such as water vapour, and/or unwanted impurities such as NO₂.
the control means are configured to order a default display of the stored pre-set dose of NO (NO_{mém}) as soon as the apparatus is switched on, i.e. as soon as it is started up, for example as soon as an On/Off button or key of the apparatus is pressed, and optionally after a latency period necessary for the initialization of the apparatus or the like, in particular after a start-up self-test aimed at ensuring that the main elements of the apparatus are functional, for example the processor(s), the operating system, etc.
the control means are configured to order a default display of the stored pre-set dose of NO (NO_{mém}) after the apparatus is started up, i.e. as soon as it is switched on, and before any effective initiation of treatment of the patient by administration of NO-containing gas.
the control means are configured to order a default display of the stored pre-set dose of NO (NO_{mém}) after the apparatus is started up, i.e. as soon as it is switched on, and before any injection of NO-containing gas into the inspiratory branch of the patient circuit.
the control means are configured to order an automatic default display of the stored pre-set dose of NO (NO_{mém}) as soon as the apparatus and the graphic display are started up.
the control means are configured to order a display of the pre-set dose of NO (NO_{mém}) in a dedicated space of the screen, typically a display window.
the control means are configured to control all the displays presented on the graphic display.
the pre-set dose of NO (NO_{mém}) is between 1 and 40 ppmv, preferably between 5 and 20 ppmv.
the pre-set dose of NO (NO_{mém}) is typically a dose of 5, 10, 15 or 20 ppmv.
the storage means are configured to store a pre-set dose of NO (NO_{mém}) of between 1 and 40 ppmv, preferably of between 5 and 20 ppmv, typically 5, 10, 15 or 20 ppmv.
it also comprises a (validation) start-of-treatment key, that is to say a selection key, actuatable (i.e. selectable) by the user, the actuation or selection of which key generates supply of a flow (i.e. flow rate) of NO-containing gas making it possible to obtain the stored pre-set dose of NO (NO_{mém}).
said at least one internal passage for conveying the flow of gas comprises one or more gas ducts, passages, pipes or the like.
the (at least one) internal passage is configured to convey the flow of NO-containing gas between (at least) a gas inlet and (at least) a gas outlet supplying the NO-containing gas, i.e. an NO/N₂ mixture.
the valve means comprise one or more valves, in particular one or more solenoid valves or one or more all-or-nothing valves.
the graphic display is a touch panel.
the graphic touch display is part of a graphical user interface (GUI).
the graphic display comprises a screen in colour or in black and white.
the control means comprise one or more microprocessors.
the microprocessor or microprocessors are arranged on an electronic board.
the control means cooperate with the valve means to control the supply of gas, in particular the flow rate of NO-containing gas, as a function of the dose of NO to be supplied, that is to say the pre-set dose of NO (NO_{mém}).
the control means cooperate with the valve means to authorize a supply of gas, in particular a flow rate of NO-containing gas, at the initiation of treatment of the patient.
conversely, the control means cooperate with the valve means to prevent or prohibit supply of gas, in particular a flow rate of NO-containing gas, before the initiation of treatment of the patient, in particular before the user presses on a start-of-treatment key.
the graphic display is configured to display information, in particular the dose of NO to be supplied, that is to say the pre-set dose of NO (NO_{mém}).
the control means are configured to order the display, on the graphic display, of the NO dose expressed in ppmv.
the graphic display is configured to additionally display a start-of-treatment key.
the start-of-treatment key is a virtual key displayed on the graphic display.
the graphic display is a touch panel.
the graphic display is configured to detect the contact of a user's finger, in particular the index finger, pressing on the surface of the graphic display, in particular on the touch panel of the graphic display.
the control means are also configured to control a display, on the graphic display, of at least one confirmation key or window, that is to say a virtual key, in response to an actuation of the start-of-treatment key (i.e. the validation key) by the user.
the control means are configured to order the valve means to provide a given flow rate of NO-containing gas in order to obtain the stored pre-set dose of NO (NO_{mém}), in response to an actuation, by the user, of at least the start-of-treatment key, in particular of at least the virtual start-of-treatment (validation) key displayed on the graphic display.
the control means are configured to order the valve means to provide a given flow rate of NO-containing gas in order to obtain the stored pre-set dose of NO (NO_{mém}), in response to successive actuations, by the user, of the start-of-treatment key and then of the confirmation key, typically virtual start-of-treatment and confirmation keys/zones, that is to say the control means order the valve means to deliver gas only after selection (e.g. pressing), by the user, of the start-of-treatment (validation) key and then selection (e.g. pressing) of the confirmation key or zone in order to confirm the NO dose and/or the start of treatment.
the control means are configured to order a display of the confirmation key within a display window displayed on the display screen.
conversely, the control means are configured to control the valve means so as not to supply a flow of NO-containing gas, in response to an actuation, by the user, of at least one treatment cancellation key or zone, in particular a virtual key or zone displayed on the graphic display, that is to say a key or zone used to refuse, cancel, etc., any treatment of the patient.
the screen is configured to display a first confirmation key or zone, the selection of which by the user (e.g. by pressing with a finger) makes it possible to confirm the proposed starting dose and to initiate the treatment, for example a key or zone designated “OK”, “GO”, “Start” or similar.
the screen is configured to display a second confirmation key or zone, the selection of which by the user (e.g. by pressing with a finger) makes it possible to cancel or stop any initiation of treatment with supply of NO, for example a key or zone designated “Stop”, “Cancel” or similar.
according to one embodiment, the screen is configured to display the second confirmation key or zone in response to the user pressing on the first confirmation key or zone.
according to one embodiment, the screen is configured to display the two confirmation keys successively, that is to say one after the other.
according to one embodiment, the screen is configured to display the two confirmation keys simultaneously, for example juxtaposed.
according to one embodiment, the screen is configured to display a confirmation window comprising the two separate confirmation keys or zones, for example a confirmation window divided into two separate zones, i.e. juxtaposed zones.
according to one embodiment, the screen is configured to display the confirmation window superimposed on the window displaying the starting dose of NO and on the start-of-treatment validation key, partially or completely masking them.
the graphic display is also configured to display, simultaneously with the confirmation window, a phrase or similar reminding the user what is to be confirmed or invalidated, for example: Start of treatment at dose of 10 ppm? or other useful information.
the confirmation window comprises tactile, i.e. virtual, confirmation keys or zones displayed on the touch screen panel.
the control means act in response to any pressing of a user's finger on one and/or the other of the virtual keys or zones displayed on the touch panel display screen.
the actuation(s) or selection(s), by the user, of the start-of-treatment key and/or of the confirmation key(s) or zone(s) is (are) effected by the user applying pressure with a finger, for example by pressing with the index finger.
the control means are configured to control a simultaneous display, on the graphic display with touch panel, of the pre-set NO dose (NO_{mém}) and of the start-of-treatment key, i.e. the virtual key for initiating/commencing treatment of the patient.
the graphic touch panel display is configured to display the pre-set dose of NO (NO_{mém}) and the start-of-treatment key juxtaposed, i.e. side by side.
the control means are configured to order the valve means to deliver a given flow rate of gas corresponding to the pre-set dose of NO (NO_{mém}) of between 5 and 40 ppmv, preferably of between 5 and 20 ppmv.
the storage means comprise a computer storage device, such as a computer memory, for example a flash memory.
the computer memory is arranged on the electronic board.
it comprises electrical power supply means, such as an electrical connection to the mains (110/220V).
it comprises a rigid housing, that is to say an outer protective shell.
the rigid housing comprises said at least one internal passage, said valve means, the graphic display and the control means.
the apparatus is configured to monitor and supply NO gas, i.e. NO-containing gas, such as an NO/N₂ mixture.
the start-of-treatment key, typically a virtual selection key, cooperates with the control means to supply them with at least one control signal for effecting supply of a flow of NO-containing gas corresponding to the stored pre-set dose of NO (NO_{mém}) desired in the final gas mixture.
the control means are configured to control the valve means in response to said control signal.
it comprises dose adjustment means, in particular configuration means, making it possible to adjust or modify the NO dose displayed by default, for example making it possible to increase or decrease the stored pre-set dose of NO (NO_{mém}) displayed on the display screen.
it comprises configuration means for adjusting or selecting and storing the desired dose of NO (NO_{mém}).
the configuration means comprise a configuration menu accessible via the display screen.
the dose adjustment means comprise one or more adjustment keys, for example “+” and “−” keys allowing the displayed dose to be increased or decreased by 1 ppm (or more).
the one or more adjustment keys are virtual keys displayed on the display screen, i.e. touch panel screen.
the one or more adjustment keys are virtual keys configured to increase or decrease the displayed dose in response to the user pressing one or other of these keys.
the dose adjustment means cooperate with the control means.
the control means modify the displayed NO dose value in response to activation of the dose adjustment means by the user, in particular in response to the user pressing a finger on one or other of these keys.

The invention also relates to an installation for administering gas to a patient, i.e. an NO-containing gas, comprising:
at least one gas source containing gaseous NO, in particular an NO/N₂ gas mixture,
a gas supply apparatus according to the invention, in particular as described above, fed with NO-containing gas by said at least one gas source, such as the NO/N₂ gas mixture,
a medical ventilator for supplying an oxygen-containing gas, such as air or an O₂/N₂ gas mixture, and
a feed line fed by the gas supply apparatus with NO-containing gas, such as the NO/N₂ gas mixture, and by the medical ventilator with oxygen-containing gas, such as air or the O₂/N₂ mixture.

Depending on the embodiment considered, the gas administration installation of the invention can comprise one or more of the following features:
the medical ventilator, that is to say a ventilation assistance apparatus, is in fluidic communication with the feed line in order to supply said feed line with a respiratory gas containing at least approximately 20% by volume of oxygen, preferably at least approximately 21% by volume of oxygen, in particular air or an N₂/O₂ mixture.
the medical ventilator is a respiratory assistance apparatus supplying the gas at constant pressure or, alternatively, the medical ventilator is an HFO (high-frequency oscillation) ventilator delivering the gas by high-frequency oscillations.
the gas feed line is fed with an NO/N₂ mixture by the gas supply apparatus and with a respiratory gas containing at least approximately 20% by volume of oxygen, preferably at least approximately 21% by volume of oxygen, preferably air or an N₂/O₂ mixture, by the medical ventilator.
the gas feed line is fed with an NO/N₂ mixture by the gas supply apparatus and with a respiratory gas containing oxygen, preferably air or an N₂/O₂ mixture, by the medical ventilator in order to form a final gas mixture for administration to the patient, containing NO, nitrogen and oxygen, or other compounds such as water vapour, and/or impurities such as argon or NO₂ species formed by oxidation of part of the NO.
the final gas mixture to be administered to the patient contains nitrogen, oxygen and NO in a proportion corresponding to the pre-set dose of NO (NO_{mém}).
the final gas mixture to be administered to the patient contains at least 20% by volume of oxygen, NO in a proportion (approximately) equal to the pre-set dose of NO (NO_{mém}), and nitrogen.
the one or more gas sources contain an NO/N₂ gas mixture containing less than 2000 ppm by volume of NO, the remainder being nitrogen, preferably less than 1000 ppm by volume of NO, the remainder being nitrogen, the therapeutic gas source contains an NO/N₂ mixture containing from 250 to 900 ppm by volume of NO, the remainder being nitrogen, for example of the order of 800 ppm by volume of NO, the remainder being nitrogen.
it further comprises a gas humidifier arranged on the gas feed line, preferably downstream of the site where the therapeutic gas supply device is fluidically connected to said gas feed line so as to feed it with therapeutic gas.
it further comprises a line for recovering the gases exhaled by the patient.
the gas feed line and the exhaled gas recovery line are connected at a connecting piece, preferably a Y-piece, and define or form all or part of a patient circuit.
the feed line forms an inspiratory branch of the patient circuit.
the exhaled gas recovery line forms an expiratory branch of the patient circuit.
the NO supply apparatus further comprises a gas analysis line fluidically connected to the gas feed line, i.e. the inspiratory branch.
the gas feed line, i.e. the inspiratory branch, comprises a flow sensor arranged between the ventilator and the site of injection of the NO-containing gas coming from the NO supply apparatus.
the flow sensor is connected to the control means of the NO supply apparatus.
the flow sensor measures the flow of oxygen-containing gas delivered by the medical ventilator and circulating in the gas feed line, i.e. the inspiratory branch.
the flow sensor returns gas flow values or signals.
the control means are configured to control the valve means to deliver the NO-containing gas flow (e.g. NO/N₂ mixture) at a given flow rate in order to obtain a dose of NO in the gas feed line corresponding to the stored dose (NO_{mém}), which given flow rate is determined from the flow of gas (e.g. air) measured by the flow sensor and from the NO concentration in the NO/N₂ mixture.
the gas feed line supplies a respiratory interface, for example a breathing mask, a tracheal intubation tube or the like.
the gas feed line, i.e. the inspiratory branch, is fluidically connected to an outlet port of the medical ventilator so as to collect and convey the gas delivered by the medical ventilator.
the exhaled gas recovery line, i.e. the expiratory branch, is fluidically connected to an inlet port of the medical ventilator so as to convey, to the medical ventilator, all or some of the gases exhaled by the patient.
at least one source of therapeutic gas comprises one or more gas containers, particularly one or more pressurized-gas cylinders.
the one or more gas containers are equipped with a gas distribution valve with or without an integrated pressure reducer (RDI).
the gas distribution valve is made of copper alloy, such as brass, and/or is equipped with a protective cowl arranged around the gas distribution valve, for example made of polymer material (i.e. plastic), metal or combinations thereof.
the one or more fluid containers are pressurized gas cylinders containing, when full, a gas mixture, in particular NO/N₂, at a pressure of at least 150 to 200 bar abs, or at least 250 to 300 bar abs.
the fluid container has a generally cylindrical shape, in particular ogival.

The invention further relates to a method for therapeutic treatment of a human, i.e. a patient, using an NO supply apparatus and/or an installation for administering gas, i.e. an NO-containing gas, comprising an NO supply apparatus for supplying said person with a gas mixture containing NO at a given dosage, said person inhaling said gas mixture, in particular a final NO-based gas mixture comprising NO, at least 20% oxygen and nitrogen, preferably at least 21% oxygen.

In the context of this treatment method:
the person suffers from acute pulmonary arterial hypertension, in particular pulmonary arterial hypertension of the newborn (PPHN), acute respiratory distress syndrome (ARDS), or pulmonary hypertension (PH) in cardiac surgery
the person is an adult, an adolescent, a child, a baby, newborn or premature.
the final gas mixture is administered via a respiratory interface, in particular a breathing mask or a tracheal intubation tube.
the final gas mixture contains nitrogen, oxygen (>21% approximately) and NO in a proportion corresponding to the pre-set dose of NO (NO_{mém}).
the final gas mixture contains less than 80 ppmv of NO, typically between 5 and 40 ppmv.
the final gas mixture is administered for 1 to several hours to 1 or several days.

In general, within the scope of the invention:
“ppmv” means parts per million by volume, -% vol. means percentage by volume.
“NO” denotes nitric oxide.
“NO₂” denotes nitrogen dioxide.
“N₂” denotes nitrogen.
“O₂” denotes oxygen.
the terms “concentration”, “dose” and “content” shall be considered as equivalents and substitutable.
the terms “means of/to/for” are considered to be wholly equivalent to and capable of being substituted by the terms “device of/to/for”, for example the term “control means” may be replaced by “control device”, the term “valve means” may be replaced by “valve device”, the “storage means” may be replaced by “storage device”, etc.

### BRIEF DESCRIPTION OF THE DRAWINGS

The invention will now be better understood from the following detailed description, given by way of illustration but without limitation, with reference to the appended figures, in which:

FIG. 1 shows an embodiment of an installation for administering gas, i.e. an NO-containing gas, comprising an NO supply apparatus according to the invention.

FIG. 2 schematically shows the operation of an NO supply apparatus according to the invention.

FIG. 3 schematically shows an embodiment of the display of the starting dose of NO on the display screen of an NO supply apparatus according to the invention, such as that of FIG. 2, in particular when it is used in an administration installation according to FIG. 1, before any supply of NO to the patient is started.

FIG. 4 schematically shows an embodiment of the configuration menu making it possible to set the starting dose of NO on an NO supply apparatus according to the invention, such as that of FIG. 2 and FIG. 3.

FIG. 5 is an example user interface showing configuration keys and a display window.

### DETAILED DESCRIPTION OF PREFERRED EMBODIMENTS

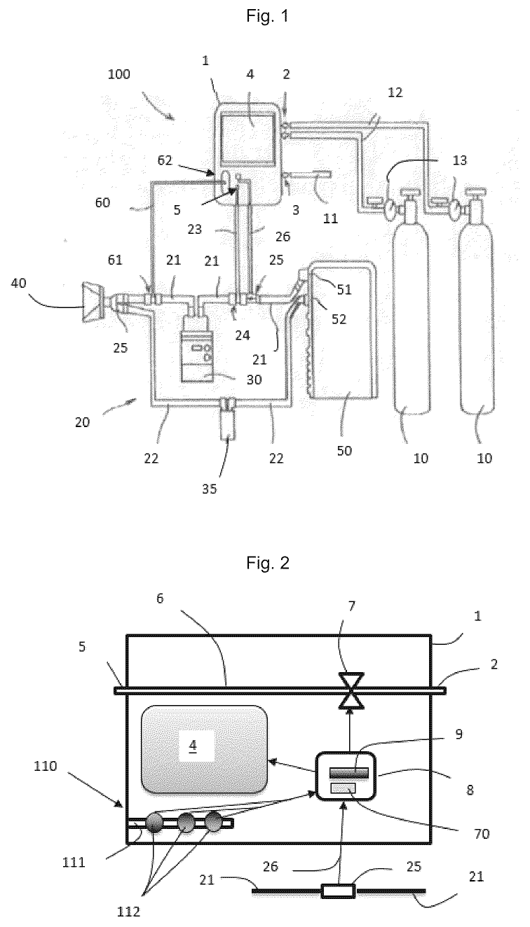

An embodiment of a gas administration installation 100 is illustrated in FIG. 1, namely for supplying an NO-based therapeutic mixture for the treatment of a patient in need thereof.

Here, the installation 100 comprises two pressurized gas cylinders as gas sources 10, each containing an NO/N₂ gas mixture containing up to 1% vol. of NO, typically between 100 and 1500 ppmv of NO (remainder N₂), namely here an NO/N₂ gas mixture containing, for example, 450 or 800 ppmv of NO (remainder N₂), which feed an NO/N₂ mixture to a device or apparatus 1 for supplying, i.e. delivering, NO, making it possible to monitor/follow and control the supply of the NO/N₂ gas mixture.

The gas cylinders 10 are fluidically connected to the NO supply apparatus 1 via gas feed lines 12, such as flexible hoses or conduits or the like, which may be equipped with devices for regulating and/or monitoring the gas pressure, such as gas regulator 13, pressure gauges, etc. The gas feed lines 12 are connected to one or more gas inlets 2 of the NO delivery device 1, which supply an internal gas passage serving to convey the gas within the NO delivery device 1, that is to say in the housing or the outer shell of the NO delivery device 1.

The NO delivery device 1 also comprises an oxygen inlet 3 fluidically connected, via an oxygen feed line 12 such as a flexible hose or the like, to an oxygen source, for example a pressurized oxygen cylinder or a hospital network, that is to say an oxygen supply line arranged in a hospital.

The installation 100 for administering gas further comprises a medical ventilator 50, that is to say a respiratory assistance apparatus, which supplies a respiratory gas flow containing oxygen, typically of the order of at least about 20% oxygen, preferably at least about 21% oxygen, such as air or an oxygen/nitrogen (N₂/O₂) mixture, or even pure oxygen in some cases.

The medical ventilator 50 and the NO supply apparatus of the installation 100 for administering gas are in fluidic communication with a gas feed line, also called an inspiratory branch 21, of a patient circuit 20. The gas feed line 21 serves to convey the gas flow to the patient, which flow is formed by mixing the oxygen-based flow (i.e. air or NO/N₂ mixture) from the medical ventilator 50 and the NO-containing flow, i.e. the NO/N₂ gas mixture, delivered by the NO supply device 1.

More precisely, the NO supply device 1 delivers or injects a given, i.e. controlled, flow of NO/N₂ mixture, for example at 450 or 800 ppmv of NO, into the gas feed line 21 via an injection line or duct 23 connected to an NO outlet 5 of the apparatus 1, in order to mix (at 24) the NO/N₂ flow with the oxygen-based gas flow (with at least approximately 20 to 21% O₂), e.g. air or an oxygen/nitrogen mixture, delivered by the medical ventilator 50 and conveyed via the inspiratory branch 21 of the patient circuit 20, so as to obtain a final mixture essentially containing NO at the desired dosage, nitrogen (N₂) and oxygen (O₂), and possibly inevitable impurities (e.g. argon, CO₂, NO₂, etc.), that is to say an NO/N₂/O₂ gas mixture.

The inspiratory branch 21 further comprises a gas humidifier 30 arranged downstream of the site 24 where NO is injected into the inspiratory branch 21. It makes it possible to humidify the gas flow, e.g. the NO/N₂/O₂ gas mixture, before it is inhaled by the patient to be treated by way of a respiratory interface 40, such as a tracheal intubation tube, a breathing mask or similar.

There is also provided a line for recovering the gases exhaled by the patient, forming the expiratory branch 22 of the patient circuit 20. The gas feed line or inspiratory branch 21 and the exhaled gas recovery line or expiratory branch 22 are connected at a connection piece 25, preferably a Y-piece.

The inspiratory branch 21 is fluidically connected upstream to an outlet port 51 of the medical ventilator 50, such as a connector, coupling or the like, so as to recover and convey the oxygen-based gas, typically air or N₂/O₂ mixture (containing approximately >20 to 21% O₂) delivered by the medical ventilator 50, while the expiratory branch 22 conveying the exhaled gases is fluidically connected to an inlet port 52 of the medical ventilator 50, such as a connector, coupling or the like, so as to return to the medical ventilator 50 all or part of the flow of the gases exhaled by the patient.

The expiratory branch 22 for the exhaled gases can comprise one or more optional components, for example a CO₂ removal device 35, i.e. a CO₂ trap, such as a hot container or the like, used to remove the CO₂ present in the patient's exhaled gases, or a filter or the like.

A flow rate sensor 25, for example of the hot wire or pressure differential type, is arranged on the gas feed line 21, between the ventilator 50 and the humidifier 30, and is connected to the NO delivery device 1 via a flow rate measurement line 26. This arrangement serves to measure the flow rate of oxygen-based gas delivered by the ventilator 50, such as air or an N₂/O₂ mixture, and circulating in the inspiratory branch 21, upstream of the site 24 where the injection duct or line 23 is connected and where the NO/N₂/O₂ gas mixture is formed. The measurements made by the flow rate sensor 25 are supplied to the control means of the NO delivery apparatus 1, where they are processed and used to control the flow rate of NO-based gas delivered, e.g. the NO/N₂ mixture.

Indeed, knowing the flow rate of oxygen-based gas makes it possible to regulate more efficiently the delivery of the NO flow (i.e. N₂/O₂) by the delivery device 1, by virtue of the flow rate measurements performed by the flow rate sensor 25 being returned, via the flow rate measurement line 26, to the control means of the delivery apparatus 1.

Regulating the flow rate of NO (i.e. N₂/O₂) makes it possible to supply an adequate proportion or amount of NO to obtain, after mixing with the flow of oxygen-based gas from ventilator 50, the desired dosage of NO in the final mixture administered to the patient, for example the stored NO concentration (NO_{mém}) being displayed by default when the appliance 1 is started up, as is explained below.

More precisely, as is shown schematically in FIG. 2, the NO supply apparatus 1 comprises, in a conventional manner, a rigid housing, for example made of polymer, through which there run one or more internal gas passages 6, such as a gas duct or the like, in order to convey the NO/N₂ flow, fed through the one or more gas feed lines 12, the latter being supplied by the NO/N₂ mixture cylinders 10.

The internal gas passage 6 fluidically connects the gas inlet (or inlets) 2 of the NO supply apparatus 1 to the injection line 23 via an NO outlet 5 of the apparatus 1, in such a way as to convey the NO-based gas flow between them. Valve means 7, i.e. one or more valve devices, for example one or more solenoid valves arranged in parallel, preferably one or more proportional (solenoid) valves, are arranged on the internal gas passage 6 in order to control the gas flow which circulates therein in the direction of the gas outlet 5 supplying the injection line 23.

The valve means 7 are controlled by control means 8, i.e. one or more control devices, also called controllers or control electronics, arranged in the housing of the NO supply apparatus 1, typically an electronic board comprising one or more microprocessors 9, preferably one or more microcontrollers, implementing one or more algorithms. They can comprise other elements, for example one or more computer memories, such as a flash memory (not shown).

The control means 8 make it possible in particular to adjust or control the flow rate of NO-based gas by controlling the valve means, typically to open or close all or some of the one or more valves, in order to obtain a determined gas flow rate which is calculated by the control means 8 on the basis of a desired NO dose value and as a function of the flow rate of oxygen-based gas (i.e. air or N₂/O₂) coming from the ventilator 50 and measured by the flow rate sensor 25 arranged on the inspiratory branch 21 and connected to the NO supply device 1 by the flow rate measurement line 26, as has already been explained.

The internal gas passage 6 of the NO supply apparatus 1 can also comprise one or more flow meters (not shown) and/or a pressure regulator, such as a pressure reducer (not shown), arranged upstream and/or downstream of the valve means 7, in order to determine the flow rate of NO-based gas circulating in the NO supply apparatus 1. The flow meter can be of the pressure differential type, the hot-wire type or some other type. It cooperates with the control means in order to provide them, once again, with measurements of the NO/N₂ flow rate, these measurements being processed by the control means 8 in order to ensure an efficient delivery of NO as a function in particular of the flow rate of O₂-based gas supplied by the medical ventilator 50.

Usually, the NO supply apparatus 1 also comprises a graphical user interface (GUI) comprising a graphical display screen 4, preferably a touch screen, i.e. a touch panel, serving to display various information items or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

The control means 8 of the NO supply apparatus 1 conventionally comprise an electronic control card and a microprocessor-based control unit 9, typically a microcontroller or the like. The control means 8 make it possible to adjust or control all the electromechanical elements of the apparatus 1. More precisely, the control card preferably integrates the control unit and is configured to control and also to analyse the signals coming from the various components, such as the sensors.

The electrical power for the NO supply apparatus 1, in particular for the components requiring electrical current in order to operate, such as the control means, the graphical display screen 4, etc., is provided conventionally by an electrical current source and/or electrical supply means (not shown), for example a connection to the mains current (110/220V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

Finally, the installation 100 also comprises a gas sampling line 60 which fluidically connects the inspiratory branch 21 to the NO supply apparatus 1. It is fluidically connected (at 61) to the gas feed line 21, between the humidifier 30 and the junction piece 25, i.e. the Y-piece, typically in the immediate vicinity of the junction piece 25, and also to an inlet port 62 of the NO supply device 1, for example a port 62 carried by a connector, coupling or the like, for connecting the gas sampling line 60, such as a flexible hose or the like, to a gas analysis line 111 equipped with sensors 112 of an internal gas analyser 110. The gas samples taken from the inspiratory branch 21 are conveyed to the NO supply device 1 where they are analysed in the internal gas analyser 110, typically one or more NO₂, NO and O₂ sensors 112, such as electrochemical cells, electrically connected to the control means 8 in order to verify the conformity of the gas samples analysed.

For obvious safety reasons, it should in fact be verified that the composition of the final gas conforms with that of the desired NO/N₂/O₂ gas mixture to be administered to the patient, in particular in order to ensure that it does not contain excessive amounts of toxic NO₂ species, that its oxygen content is not hypoxic, that it does not have an excessively high NO₂ content, and that its NO content corresponds to the desired dosage, i.e. the dose of NO to be administered by inhalation that is usually chosen by the healthcare personnel, i.e. a doctor or the like.

As has already been explained, the starting dose of NO is usually chosen, i.e. adjusted or selected, by the medical personnel at the level of the NO supply device used, after the NO supply device has been started up. Thus, when an NO supply device is started up, i.e. switched on, the display 4 displays an NO content or dose equal to 0 ppm, and the healthcare provider must then set what is called the “starting” dose of NO, which is the one desired at the beginning of the treatment.

In other words, when it is started up, an NO supply apparatus 1 according to the prior art displays what is called a zero “starting” dose (i.e. 0 ppm), that is to say a concentration of NO equal to 0 ppm, and the healthcare provider sets a desired “starting” dose, for example 5, 10, 15 or 20 ppm. for example by intervening at the user interface of the apparatus in order to enter the dose or select it from a menu displaying different possible dose choices or similar.

It will therefore be understood that NO dosing errors can sometimes occur as a result of errors that are made when setting or selecting the starting dose. Medical personnel often need to act urgently when starting NO treatment on a patient, for example a hypoxic newborn baby, and may therefore make a mistake when entering or selecting the dose of NO to be administered at the start.

It will be readily understood that any dose error, i.e. too large a dose or too small a dose, can have serious consequences for the patient, especially since people requiring inhaled NO treatment are often weak and/or in a serious medical condition, for example newborn babies with severe hypoxaemia.

According to the invention, the NO supply device 1 has been modified to reduce the risk of the patient receiving an inappropriate concentration or dose, therefore to increase the safety of NO treatment by avoiding the NO dosing errors that can occur at the beginning of treatment, that is to say the errors related to fixing the “starting” dose of NO, that is to say just before the commencement of the administration of NO to the patient, i.e. the start of treatment of the patient.

To do this, according to the invention, the NO supply device 1 is provided with storage means 70 configured to store a pre-set non-zero dose of NO (NO_{mém}), that is to say a dose provided and decided on in advance, typically a dose of between 1 and 40 ppm, for example a dose of 10, 15, 20 ppm or the like, which will be what is called the starting dose.

Moreover, the control means 8 including the microprocessor 9, which cooperate with the storage means 70, are configured to order a display (at 71), on the graphic display 4, of the pre-set dose of NO (NO_{mém}) stored as the starting dose. The storage means 70, for example a flash memory or the like, may be arranged on the electronic board carrying the microprocessor 9 and connected to the latter.

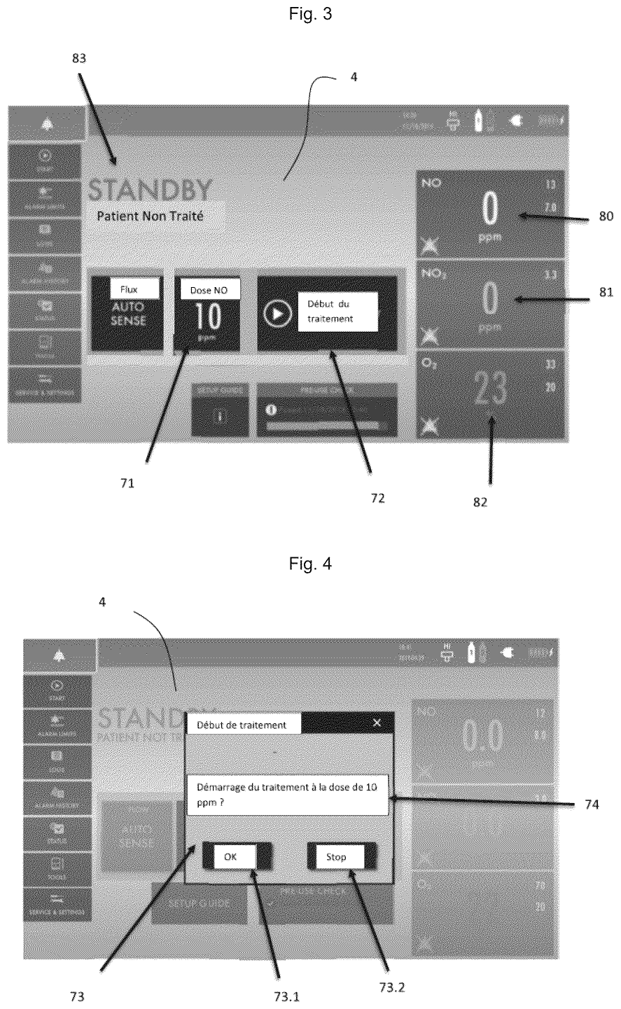

In other words, the control means 8, typically the microprocessor 9, control or order the graphic display 4 to present a default display of the stored pre-set dose of NO (NO_{mém}) as soon as the apparatus 1 is started up, that is to say as soon as it is switched on, within a dedicated space of the screen 4, typically a display window 71 or the like. For example, the graphic display 4 can display (at 71) a starting dose value of “10 ppm”, as illustrated in FIG. 3.

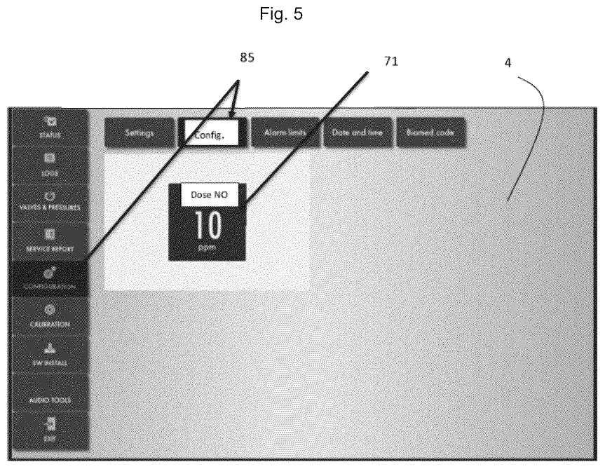

The stored starting dose can be fixed once and for all by the medical personnel, for example by a member of the healthcare team designated by the hospital department and preferably in the presence of a person certified by the manufacturer of the apparatus, via a menu and/or one or more configuration keys 85 of the apparatus 1 for example, as is shown schematically in FIG. 5, and can then be recorded by the storage means 70, for example a flash memory or the like. This dose can optionally be modified subsequently, via the configuration menu and/or the configuration key(s) 85, if the medical personnel so desire, and the new starting dose will then be stored in the same way.

Furthermore, the apparatus 1 comprises a start-of-treatment key 72 actuatable by the user, the actuation of which key generates a supply of the flow of NO-containing gas, that is to say a controlled NO flow rate, in order to obtain the stored pre-set dose of NO (NO_{mém}) in the final mixture administered by inhalation to the patient.

Advantageously, the graphic display 4 is a touch panel, typically a colour touch pad, and the start-of-treatment key 72, that is to say the treatment validation key, is a virtual key displayed on the graphic display 4, that is to say a touch key displayed on the touch screen, as is illustrated in FIG. 3.

When a healthcare provider presses with a finger, for example an index finger, on the key 72 for validating the start of treatment, this pressure will be recognized by the touch panel, and a corresponding signal will be transmitted to the control means 8, typically to the microprocessor 9, which will then order the valve means in particular to start treatment with inhaled NO by sending a flow of NO into the inspiratory branch 21 of the patient circuit 20, where it mixes (at 24) with the flow of oxygen-containing gas coming from the ventilator 50, such as air or an O₂/N₂ mixture, and thereby obtain the desired final gas mixture containing the desired NO dosage, namely the stored pre-set dose of NO (NO_{mém}), here 10 ppm, at the beginning of the treatment.

Of course, this dose can be modified subsequently depending on the patient's response to treatment with inhaled NO, that is to say increased or decreased by the medical personnel. This can be done at any time, before the start of the treatment and, thereafter, throughout the duration of the treatment.

The example in FIG. 3 shows how the screen 4 can look after it has been switched on by the medical personnel, but before any administration of NO. The stored pre-set dose of NO (NO_{mém}), here 10 ppm, is displayed by default in the display window 71, and the start-of-treatment validation key 72 is also displayed, just next to the display window 71.

It will be seen in FIG. 3 that, despite the starting dose of 10 ppm being displayed, the administration of NO has not yet started, that is to say the device 1 is not yet injecting a flow of NO, typically an NO/N₂ mixture, at a controlled flow rate into the inspiratory branch 21 of the patient circuit 20, since the graphic display 4 also displays zero NO and NO₂ contents (i.e. 0 ppm) in dedicated windows 80, 81. On the other hand, it will be seen that the ventilator 50 already delivers the flow of oxygen-containing gas (>20% vol), since a dedicated window 82 displays an oxygen (O₂) content of 23% here. These concentrations of NO, O₂ and NO₂ are determined by the internal gas analyser 110, namely the gas analysis line 111 equipped with NO₂, NO and O₂ sensors 112, such as electrochemical cells, electrically connected to the control means 8, as has been explained above.

The fact that the treatment has not yet begun is also confirmed by an additional display of information relating to the treatment (i.e. in progress or not in progress) intended for the medical personnel, in a dedicated space 83 of the screen 4, namely here the following information: STANDBY-Untreated Patient, or similar information.

Of course, the information relating to the treatment changes as soon as the patient care provider presses the start-of-treatment key 72, i.e. it is updated. The screen 4 can then display new information such as: TREATMENT IN PROGRESS-Treated Patient, or similar.

The correct course of the treatment can then be verified by the medical personnel by consulting in particular the dedicated windows 80, 81, 82 which then display non-zero values of NO and O₂. For its part, the NO₂ content must be as low as possible, preferably (near) zero, i.e. about 0 ppm, because NO₂ species (which result from oxidation of a small part of the NO by oxygen) are toxic and must be minimized/avoided.

According to an advantageous embodiment, the control means 8 are also configured to control a display, on the graphic display 4, of one or more confirmation keys or windows 73, that is to say of one or more other virtual keys or the like, in response to an actuation of the start-of-treatment key 72 by the user, as illustrated in FIG. 4.

Said one or more confirmation keys or windows 73 allow the medical personnel to confirm or cancel a start of treatment of the patient with the stored pre-set dose of NO (NO_{mém}), for example 10 ppm in the example of FIG. 3.

To do this, depending on the embodiment, it is possible to provide either two separate confirmation keys 73, i.e. juxtaposed, or, as is illustrated in FIG. 4, a confirmation window 73 divided into two separate juxtaposed zones 73.1, 73.2:
of which one 73.1 (i.e. first zone) is used to confirm the proposed starting dose, i.e. 10 ppm here, and to initiate the treatment, for example a key or zone 73.1 labelled “OK”, “GO” or “Start” or any similar term; and
of which the other 73.2 (i.e. second zone) is used to cancel or stop any start of treatment with supply of NO, for example a key or zone 73.2 labelled “Stop” or “Cancel” or any similar term.

Confirmation or cancellation (i.e. non-confirmation) is preferably carried out by a healthcare provider pressing a finger (e.g. an index finger) on one or other of these separate zones or keys 73.1, 73.2.

As is illustrated in FIG. 4, provision can be made that the screen or graphic display 4 displays, in the foreground, a confirmation window 73 superimposed on the window 71 displaying the starting dose of NO and on the start-of-treatment validation key 72, partially or completely masking them. The background of the screen 4 can then be greyed out.

Moreover, the graphic display 4 can also be configured to simultaneously display a phrase of some kind (at 73) reminding the user what he or he has to confirm or invalidate, namely here: Start treatment at dose of 10 ppm? or other information.

Thanks to the invention, no dose error is possible at the start-up of the apparatus 1, because the stored NO dose is proposed, that is to say is displayed by default (at 71) on the graphic display 4 as soon as the apparatus 1 is started up, hence before any NO has been supplied to the inspiratory branch 21 of the patient circuit 20 and therefore to the patient. The supply of NO then begins only after this proposed NO has been validated by the medical personnel, or even only after confirmation of the treatment via the one or more confirmation keys or windows 73.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing (i.e., anything else may be additionally included and remain within the scope of “comprising”). “Comprising” as used herein may be replaced by the more limited transitional terms “consisting essentially of” and “consisting of” unless otherwise indicated herein.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. An apparatus (1) for supplying an NO-containing gas, comprising:
at least one internal passage (6) for conveying a flow of NO-containing gas,
valve (7) arranged on said at least one internal passage,
a graphic display (4), and
a control (8) with a microprocessor (9) cooperating at least with the valve (7) to control the supply of gas,
characterized in that:
the apparatus also comprises storage (70) adapted for storing a pre-set non-zero dose of NO (NO_{mém}) to be administered by inhalation to a patient at the initiation of treatment by inhaled NO,
the control (8) is configured to control a display, on the graphic display (4), of the pre-set dose of NO (NO_{mém}) that has been stored, and
the graphic display (4) is configured to present, before any initiation of the treatment by inhaled NO, a default display (at 71) of the pre-set dose of NO (NO_{mém}) that has been stored.

2. The apparatus according to claim 1, characterized in that the apparatus further comprises a start-of-treatment key (72), actuatable by a user, the actuation of which key is configured to start a supply of the flow of NO-containing gas in order to obtain the stored pre-set dose of NO (NO_{mém}).

3. The apparatus according to claim 2, characterized in that the graphic display (4) is a touch screen, and the start-of-treatment key (72) is a virtual key displayed on the graphic display (4).

4. The apparatus according to claim 3, characterized in that the control (8) is further configured to control a display, on the graphic display (4), of at least one confirmation key or window (73) in response to an actuation of the start-of-treatment key (72) by the user.

5. The apparatus according to claim 3, characterized in that the control (8) is configured to control the valve (7) to provide a given flow rate of NO-containing gas in order to obtain the stored pre-set dose of NO (NO_{mém}), in response to an actuation, by the user, of of at least the virtual start-of-treatment key (72) displayed on the graphic display (4).

6. The apparatus according to claim 4, characterized in that the control (8) is configured to control the valve (7) to provide a given flow rate of NO-containing gas in order to obtain the stored pre-set dose of NO (NO_{mém}), in response to successive actuations, by the user, of the start-of-treatment key (72) and then the confirmation key or window (73).

7. The apparatus according to claim 6, characterized in that the user actuation or actuations of the start-of-treatment key (72) and/or of the confirmation key or window (73) are configured to be effected by the user pressing on them with a finger.

8. The apparatus according to claim 1, characterized in that the storage (70) is configured to store a pre-set dose of NO (NO_{mém}) of between 1 and 40 ppmv.

9. The apparatus according to claim 1, characterized in that the control (8) is configured to control a display of the stored pre-set dose of NO (NO_{mém}) as soon as the apparatus (1) is switched on, but before any start of treatment.

10. The apparatus according to claim 8, characterized in that the storage (70) is configured to store a pre-set dose of NO (NO_{mém}) of 5, 10, 15 or 20 ppmv.

11. The apparatus according to claim 2, characterized in that the graphic display (4) is configured to display a first confirmation key or zone, the selection of which by the user, by pressure of a finger, makes it possible to confirm the proposed starting dose and to initiate the treatment with supply of NO.

12. The apparatus according to claim 11, characterized in that the graphic display (4) is configured to display a second confirmation key or zone, the selection of which by the user, by pressure of a finger, makes it possible to cancel or stop any initiation of treatment with supply of NO.

13. The apparatus according to claim 12, characterized in that the graphic display (4) is configured to display the second confirmation key or zone in response to the user pressing on the first confirmation key or zone.

14. An installation (100) for administering an NO-containing gas to a patient, comprising:
at least one gas source (10) containing an NO/N₂ gas mixture,
a gas supply apparatus (1) according to claim 1, connected to and configured to be fed with NO-containing gas by said at least one gas source (10),
a medical ventilator (50) for supplying an oxygen-containing gas, and
a feed line (20, 21) fed by the gas supply apparatus (1) with the NO-containing gas, and by the medical ventilator (50) with the oxygen-containing gas.

15. The installation according to claim 14, characterized in that said at least one gas source (10) contains an NO/N₂ gas mixture containing less than 2000 ppmv of NO, the remainder being nitrogen.
